(19) United States
(12) Patent Application Publication
(10) Pub. No.: US 2022/0265252 A1
(43) Pub. Date: Aug. 25, 2022

(54) DEVICE FOR TISSUE HARVESTING FOR BIOPSY EXAMINATION

(71) Applicant: Covidien LP, Mansfield, MA (US)

(72) Inventor: Olesea Diaz-Chiosa, Naugatuck, CT (US)

(21) Appl. No.: 17/180,524

(22) Filed: Feb. 19, 2021

(51) Int. Cl. A61B 10/04; A61B 10/06; A61B 10/00; A61B 34/30

(52) U.S. Cl. CPC A61B 10/04; A61B 10/06; A61B 10/0096; A61B 34/30; A61B 2010/0208

(57) ABSTRACT

A suction dissection catheter and method of dissecting a lymph node including forcing gripping fingers on the suction dissection catheter over a portion of the lymph node, applying a vacuum, applying energy to a garter electrode connecting the gripping fingers and dissecting the lymph node from the tissue surrounding the lymph node.

### FIELD

This disclosure relates to the field of biopsy and tissue harvesting, and particularly to biopsy and tissue harvesting of lymphatic tissue for the determination of potential disease spread.

### BACKGROUND

The disclosure relates to the field of medical devices and methods used in the diagnoses of diseases such as cancer which have the ability to metastasize within a patient's body. More specifically, the disclosure is directed to methods and devices for accessing sentinel lymph nodes associated with a lesion site within a patient's body so that they may be selectively removed and analyzed to determine whether disease has spread from the primary lesion site to the sentinel lymph nodes.

Metastasis, or migration of cancerous cells, typically occurs through lymph ducts. Sentinel lymph nodes are so-called because, where metastasis occurs, such lymph nodes are often the first locations to harbor metastatic cancer cells. These lymph nodes thus serve as sentinels warning of the spread of the cancerous lesion. A sentinel lymph node may be identified by injection of radioactive material into a primary lesion site such as a cancerous tumor. Detection of radiation at a location other than the injection site indicates that migration of the radioactive material has occurred. The first lymph nodes into which the radioactive material migrates are thus identified as the sentinel lymph nodes.

The determination of the severity of the disease or staging is frequently determined by the level a lymph node involvement in those lymph nodes correspond to the primary cancer lesion site in the breast. The sentinel lymph nodes can be in fluid communication with other surrounding lymph nodes, however, lymph drainage from the lesion site will first flow to the sentinel lymph nodes. Thereafter, lymph fluid drainage may then continue on to lymph nodes surrounding the sentinel nodes.

Studies have shown that by the time a typical cancer lesion reaches the size of 1-2 cm, the cancer will have metastasized to at least one of the sentinel lymph nodes in about one third of patients. Malignant cells break off and drain through the lymph fluid ducts to the lymph nodes and will be apparent in excised lymph nodes if the malignant cells embed in the lymph node. In patients with more advanced disease, the likelihood of spread to sentinel nodes is higher as is the likelihood of spread of the disease to the lymph nodes surrounding the sentinel lymph nodes.

It should be noted that traditional lymph node dissection techniques can be painful and debilitating procedure for patients who often suffer from severe lymph edema as a result of the body's inability to channel the flow of lymph fluid once most or all of the lymph nodes have been excised. Further, many lymph nodes are surrounded by critical structures including blood vessels, related lymph ducts, and other tissues making the procedure quite challenging for the surgeons.

Radioactive materials have been used as localizing agents which can be injected into the area of a primary lesion to monitor the flow of the materials within the patient's body using a variety of detectors. A pharmaceutically-acceptable solution containing a radioactive material may be termed a radiopharmaceutical. Suitable radioactive materials include the radioactive elements Technetium 99, Indium 111, iodine 123 or Iodine 125.

Although techniques exist to locate the sentinel lymph nodes of a patient with such radiopharmaceutical tagging, what has been needed are methods and devices to precisely access the sentinel lymph nodes of the patient while minimizing pain and the surgical difficulties associated with the neighboring tissue structures. Therefore, there is a need in the art for an improved system and method for the capturing of body tissue samples. More particularly, there is a need for a system and method for capturing thoracic lymph nodes suspected of being cancerous.

### SUMMARY

One aspect of the disclosure is directed to a suction dissector including: a handle configured with a plurality of controls. The suction dissector also includes a vacuum source in fluid communication with the handle and operable with one of the plurality of controls; a shaft in fluid communication with the vacuum source and connected to the handle. The suction dissector also includes a gooseneck formed of articulating joints and connected to a distal end of the shaft, the gooseneck configured to articulate in at least one direction; a plurality of gripping fingers flexibly connected to a distal end of the gooseneck. The suction dissector also includes a garter electrode interconnecting the plurality of gripping fingers, the garter electrode being sized to be received over a lymph node and biasing the gripping fingers in a closed position. The suction dissector also includes an energy source electrically connected to the garter electrode, where the garter electrode is configured to cut and cauterize tissue in contact with the garter electrode. The suction dissector also includes a plurality of first pull wires configured to articulate the gooseneck and operable with one of the plurality of controls. The suction dissector also includes a plurality of second pull wires configured to move the gripping fingers from the closed position to an open position and operable with one of the plurality of controls. The suction dissector also includes a pathology container in fluid communication with the vacuum source and the shaft, where upon dissection the lymph node by application of energy generated by the energy source to the garter electrode to cut and cauterize tissue surrounding the lymph node, the lymph node is transported along the gooseneck and shaft by the vacuum and received in the pathology container.

Implementations of this aspect of the disclosure may include one or more of the following features. The suction dissector where the first and second pull wires are motor driven. The suction dissector further including at least one second pull wire to open or close the of gripping fingers. The suction dissector where the at least one second pull wire is operably connected to motor to extend and retract the at least one second pull wire. The suction dissector where the at least one second pull wire is robotically actuated. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

A further aspect of the disclosure is directed to a suction dissector including: a vacuum source, a shaft in fluid communication with the vacuum source. The suction dissector also includes a gooseneck formed of a plurality of articulating joints and connected to a distal end of the shaft, the gooseneck configured to articulate in at least one direction; a plurality of gripping fingers flexibly connected to a distal end of the gooseneck. The suction dissector also includes a garter electrode interconnecting the plurality of gripping fingers and sized to be received over a lymph node. The suction dissector also includes an energy source electrically connected to the garter electrode, where application of energy to the garter electrode cuts and cauterizes tissue in contact with the garter electrode. The suction dissector also includes a pathology container in fluid communication with the vacuum source and the shaft, where upon dissection the lymph node is transported along the shaft by the vacuum and received in the pathology container.

Implementations of this aspect of the disclosure may include one or more of the following features. The suction dissector further including at least one first pull wire to articulate the gooseneck. The suction dissector where the at least one first pull wire is operably connected to motor to extend and retract the at least one first pull wire. The suction dissector where the at least one pull wire is robotically actuated. The suction dissector where the garter electrode applies a spring force against the gripping fingers to move the gripping fingers into a closed position. The suction dissector where the gripping fingers are formed such that they apply a spring force relative to the shaft to move the gripping fingers into a closed position. The suction dissector further including a spring associated with the gripping fingers to move the gripping fingers into a closed position. The suction dissector where the pathology container is removable and replaceable. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

Yet a further aspect of the disclosure is directed to a method of dissecting a lymph node including: navigating a distal portion of a suction dissection catheter to a lymph node within a patient, forcing one or more gripping fingers secured to a distal end of the suction dissection catheter over a portion of the lymph node, applying a vacuum through a shaft of the suction dissection catheter, applying energy from an energy source to a garter electrode connecting the one or more gripping fingers to tissue surrounding the lymph node such that the tissue surrounding the garter electrode is cut an cauterized, dissecting the lymph node from the tissue surrounding the lymph node, drawing the dissected lymph node through the shaft via the applied vacuum, and capturing the dissected lymph node in a pathology container. Other embodiments of this aspect include corresponding computer systems, apparatus, and computer programs recorded on one or more computer storage devices, each configured to perform the actions of the methods and systems described herein.

Implementations of this aspect of the disclosure may include one or more of the following features. The method further including determining a location of one or more lymph nodes; The method where the determining is via application of a radioactive substance to a lesion and observing transmission of the radioactive substance to one or more lymph nodes. The method where the navigation is performed via a robotic interface with the suction dissection catheter. The method where the energy is radio frequency energy. The method where application of force on the gripping fingers causes them to expand to extend around the lymph node and easing of the force allows the garter electrode to retract the gripping fingers. The method where a spring force associated with one or more of the garter electrode, a material of the gripping fingers, or a spring incorporated into the interface between the gripping fingers and a gooseneck at the end of the shaft urge the gripping fingers together. Implementations of the described techniques may include hardware, a method or process, or computer software on a computer-accessible medium, including software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

### BRIEF DESCRIPTION OF THE DRAWINGS

Various exemplary embodiments are illustrated in the accompanying figures. It will be appreciated that for simplicity and clarity of the illustration, elements shown in the figures referenced below are not necessarily drawn to scale. Also, where considered appropriate, reference numerals may be repeated among the figures to indicate like, corresponding or analogous elements.

FIG. 1 is a perspective view of a suction dissector in accordance with the disclosure;

FIG. 2 is a perspective view of a distal portion of the suction dissector of FIG. 1 in accordance with the disclosure;

FIG. 3A is a perspective view of gripping fingers and distal end of a shaft of the suction dissector of FIG. 1 in accordance with the disclosure;

FIG. 3B is a bottom view of the gripping fingers of FIG. 3A with garter electrodes spanning between the gripping fingers in accordance with the disclosure;

FIG. 4 is a perspective view of a distal portion of the suction dissector of FIG. 1 depicting a lymph node having been dissected in accordance with the disclosure;

FIG. 5A is a perspective view of a distal portion of the suction dissector of FIG. 1 depicting the travel of a dissected lymph node through the gooseneck and shaft in accordance with the disclosure;

FIG. 5B is a profile view of a proximal end of the suction dissector of FIG. 1 with a pathology container affixed thereto in accordance with the disclosure;

FIG. 5C is a profile view of a pathology container with a dissected lymph node therein ready for shipment to a pathology lab in accordance with the disclosure; and

FIG. 6 is a flow chart detailing a lymph node dissection procedure in accordance with the disclosure.

### DETAILED DESCRIPTION

This disclosure is directed to a radio frequency lymph node dissector employing a garter electrode. The garter electrode is expandable by two or more gripping fingers between which garter electrode extends. Placement of the garter electrode over the lymph node by the gripping fingers minimizes the margin around the lymph node needed for dissection. Application of RF energy causes the garter electrode to cut and cauterize the tissue surrounding the lymph node. As the garter electrode cuts, the gripping fingers extend further into the tissue until the gripping fingers are able to fully encapsulate the lymph node. The final cutting applied by the RF garter electrode severs the lymph node from the surrounding tissue. Vacuum is applied via a catheter to which the gripping fingers are attached. The vacuum also assists in displacement and eventual removal of the lymph node from the surrounding tissue. Via the cutting and cauterization process, any small blood vessels or other ducts in the tissue surrounding the lymph node may be cauterized and effectively sealed preventing blood loss. Larger blood vessels function as a heat sink due to their flow volume and are largely unaffected by the process. Once dissected by the garter electrode, the vacuum evacuates the lymph node through the catheter to a sample collection device, where the lymph node may be captured in a hands-free manner, catalogued, and send to pathology for analysis. In this manner lymph nodes may be collected from the patient, particularly in difficult to reach areas such as the thoracic cavity and the lungs, where manual harvesting methods may be challenging. Further such harvesting can be conducted in a largely bloodless manner with minimal pain and post-surgical complications for the patient. These and other aspects of the disclosure are described in greater detail below.

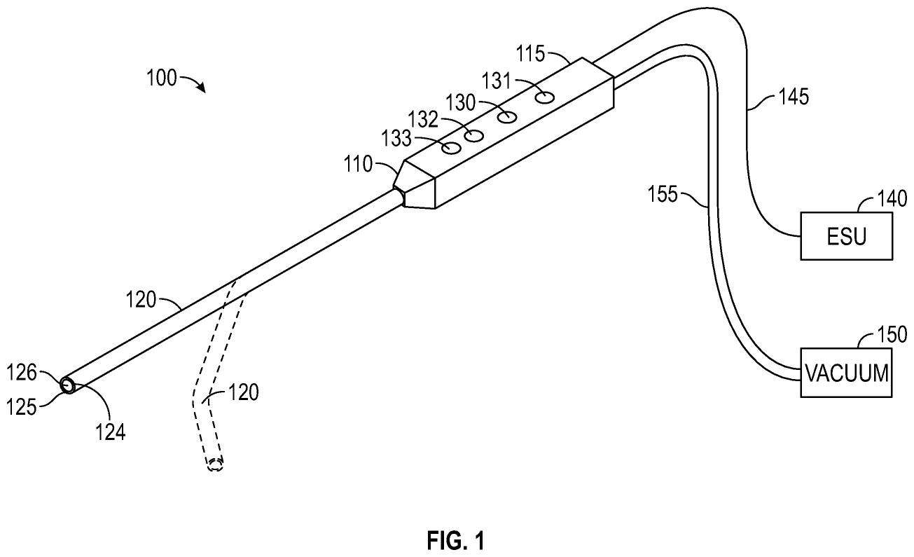

With reference to FIG. 1, an electrosurgical lymph node dissector 100 is presented including a having a suction dissector 110 that is operably coupled to an electrosurgical generator 140 via a conductor 145. Suction dissector 110 is operably coupled to a vacuum source 150 by a lumen 155. Suction dissector 110 includes a handle 115 disposed at the proximal end thereof and an elongated shaft 120 extending distally from the handle 115. The shaft 120 may be formed from material having malleable or flexible properties, for example without limitation, metallic material such as aluminum and alloys thereof and/or polymeric materials such as polyurethane (PU) or polyvinyl chloride (PVC). A shaft 120 thus formed may be bent to a desired shape by the user, as shown by way of example by bent shaft 120′. Additionally or alternatively, the shaft 120 may include a flexible gooseneck as described in greater detail below which may include one or more pull wires to enable articulation of the shaft 120 to align a distal portion of the shaft 120 with a lymph node.

Distal end 124 of shaft 120 includes an exposed electrode 125 (e.g., the garter electrode described briefly above) for delivering electrosurgical energy to tissue. A lumen 126 defined longitudinally through the shaft 120 provides suction to a surgical site. Lumen 126 is in fluid communication with vacuum source 150 via lumen 155.

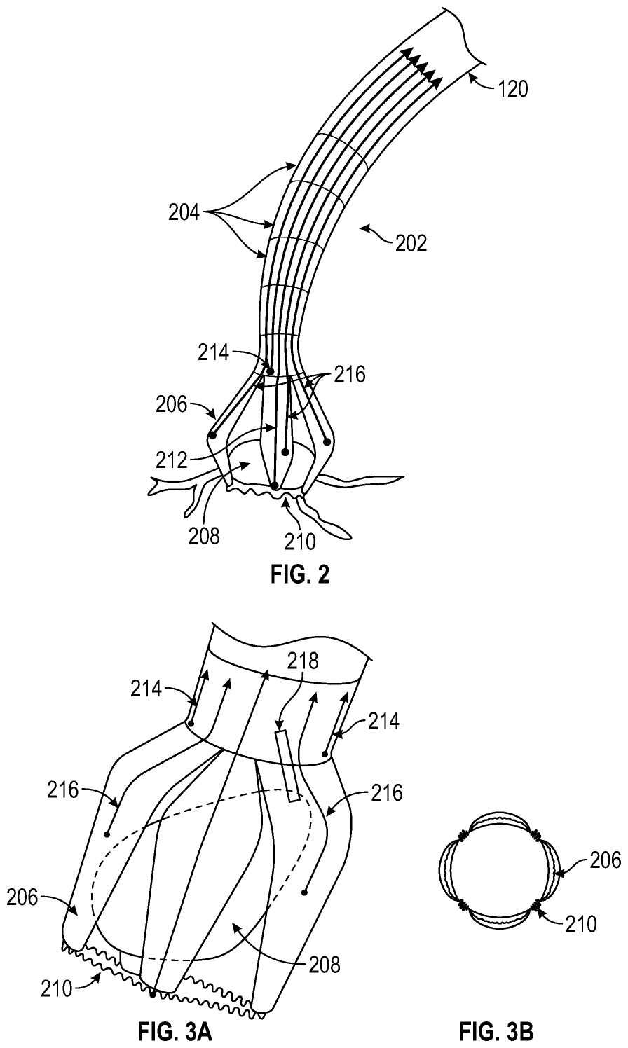

In an embodiment, handle 115 may include a control 130 which may be a hand switch for controlling the application of electrosurgical energy, i.e., activation and deactivation of an electrosurgical signal. Handle 115 may include an additional or second control 131 for controlling the application of suction to the surgical site. In embodiments, control 131 may be operably coupled to a valve (not shown) that may be disposed within handle 115, shaft 120, vacuum source 150, and/or lumen 155. In other envisioned embodiments, control 131 may be operably coupled to a regulator, motor control, or other suitable manner of vacuum control. A third control 132 may be operably connected to an articulating mechanism (e.g., a pull wire) to achieve articulation of the distal portion of the shaft 120. Additionally or alternatively, a further control 133 may be operably connected to one or more pull wires to open and close a plurality of gripping fingers (FIG. 2, 206). The controls 130-133 may be a manual control such as a slide, or an electrical control such as a toggle, switch, or other means for electrically or electromechanically enabling one or more of the functions of the controls 130-133.

The electrode 125 is employed to apply a radiofrequency (“RF”) signal to the desired tissue to dissect a lymph node in accordance with the disclosure. For example, RE energy in the form of an alternating current electrical signals in approximately the 200 kHz-3.3 MHz range may be generated by the electrosurgical generator 140, to cut or coagulate biologic tissue. This signal can be a sinusoidal waveform operating in a continuous mode at a 100% duty cycle, or pulse modulated at a duty cycle of less than 100%. Typically, electrosurgical signals are operated at 100% duty cycle for maximal cutting effect, and are pulse modulated at duty cycles ranging from 50% to 25% for less aggressive cutting, or, at a substantially lower duty cycle of approximately 6%, for coagulating tissue. The electrosurgical carrier signal may also be varied in intensity. The electrosurgical signal is applied to the patient via electrodes in either monopolar mode, or bipolar mode. In monopolar mode, the active electrode is the surgical instrument at the surgical site, and the return electrode is elsewhere on the patient, such that the electrosurgical signal passes through the patient's body from the surgical site to the return electrode. In bipolar mode, both the active and return electrodes are at the surgical site, such as with an instrument having an array of electrodes, so that the electrosurgical signal passes only through the tissue situated between the RF electrodes of the instrument.

FIG. 2 depicts aspects of a distal portion of the shaft 120. As noted above, the distal portion of the shaft 120 may include a flexible gooseneck 202. The gooseneck 202 may be formed of a desired number of articulating joints 204. These articulating joints 204 can move relative to each other and the shaft 120 such that an ordinarily straight shaft 120 can achieve a curved shape as depicted in FIG. 2. At the distal end of the flexible gooseneck 202 are a plurality of gripping fingers 206. The gripping fingers 206 are sized and shaped to be placed over a lymph node 208. The lymph node 208 may be for example formed in an airway wall of the lungs, or in another location within the thoracic cavity. Extending between the gripping fingers 206 is a garter electrode 210. As will be appreciated, an electrode wire 212 extends from the garter electrode 210, through the length of the shaft 120 and to the electrosurgical generator 140. This electrode wire 212 may be imbedded into the wall of the shaft 120, or may be secured to an inner side or outer side of the shaft 120. In one embodiment the gripping fingers 206 are formed of a conducting material such as a metal and may optionally be coated with an insulating material. When the gripping fingers 206 are coated with an insulating material the garter electrode 210 is exposed to enable transfer of energy from the garter electrode 210 to the tissue to cut and coagulate the tissue around the lymph node 208.

The garter electrode 210, as depicted in FIGS. 3A and 3B is formed such that it is expandable or retractable (e.g., a metal spring). This can be seen in FIG. 3B where the portion of the garter electrode 210 between the gripping fingers 206 takes on the shape of a compressed spring. Alternatively, as depicted in FIG. 3A, the garter electrode appears as an expanded spring (e.g., a wire) extending between the gripping fingers 206.

In one embodiment of the disclosure, after navigating the distal portion of the suction dissector 110 such that the gripping fingers 206 at least partially surround the lymph node 208 application of force on the proximal portion of the suction dissector (e.g., the handle 115) causes the gripping fingers to expand or flare outward when forced against the tissue of the patient. RF energy from the generator 140 is applied to the tissue around the lymph node 208 via the garter electrode 210. The garter electrode 210 cuts and coagulates tissue surrounding the lymph node 208 by application of the RE energy. Continued application of pressure on the handle 115 causes the garter electrode 210 to continue to cut into the tissue. Once a desired depth is reached or a desired time allowed for cutting, the pressure is released on the handle 115. Release of the pressure will cause the spring force in the garter electrode, or other inward biasing of the gripping fingers 206 to allow the gripping fingers 206 to bias towards one another. This biasing action along with continued application of RF energy continues the cutting an coagulation of the tissue surrounding the lymph node 208 until the lymph node 208 is excised from the surrounding tissue and received into the lumen 126 of the shall 120.

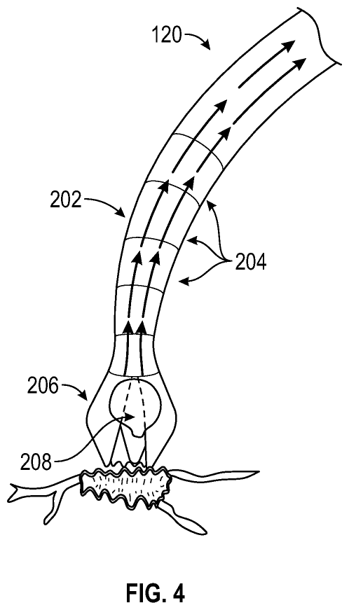

The vacuum 150 generates vacuum that is transmitted through the lumens 155 and 126. This vacuum initially assists in drawing the lymph node 208 into the gripping fingers 206 and the lumen 126 (which extends through the gooseneck 202). By drawing the lymph node 208 away from the surrounding tissue, the cutting and coagulation process, described above, is eased and the margins around the lymph node 208 reduced. Once the garter electrode 210 and other biasing means for the gripping fingers 206 have returned to their original shape, the lymph node 208 has been dissected from the surrounding tissue as shown in FIGS. 3A and 4. At this time RF energy application can be stopped. The remaining tissue site from which the lymph node was dissected is now cauterized and substantially free from bleeding or the leaking of other bodily fluids.

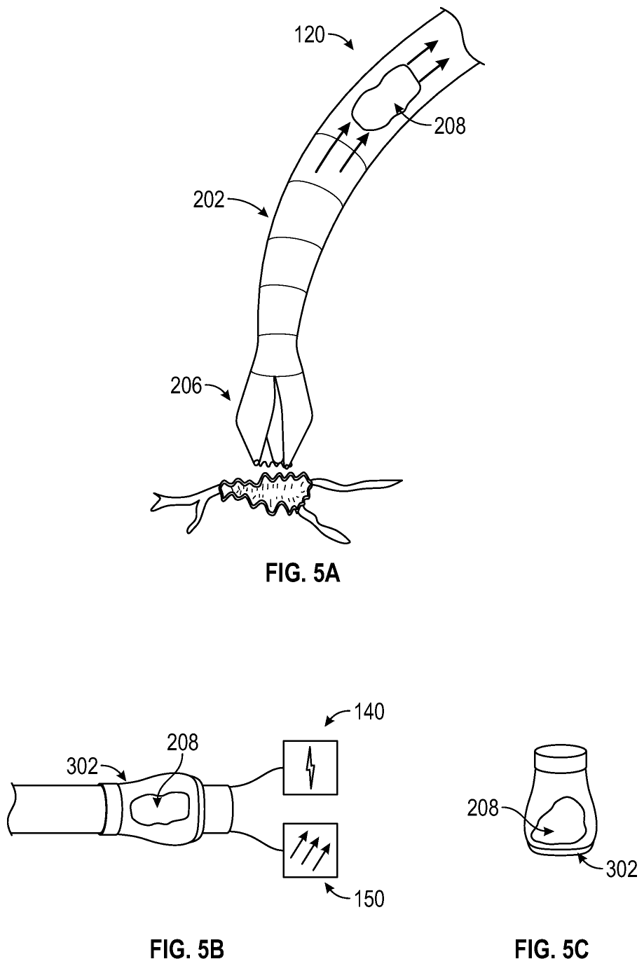

As depicted in FIG. 5A, the continued application of vacuum causes the dissected lymph node 208 to traverse the gooseneck 202, and the lumen of shaft 120 of the suction dissector 110. On the proximal end of the suction dissector and in fluid communication with both the lumen of shaft 120 and the lumen 155, is a pathology container 302. The pathology container may be removably affixed to the handle 115 of the suction dissector. After traversing the lumen of shaft 120, the pathology container 302 receives the lymph node 208 (or other dissected tissues). The pathology container 302 may then be sealed. The pathology container 302 may be labeled with the or appropriate level for the dissected lymph node 208 as well as other indications of the location from which it was excised (e.g., right or left), the patient's name, and other relevant information. The pathology container 302 may then be sent to a pathologist for study and determination of whether it contains cancerous cells which indicate spread of the disease.

After removal of a single lymph node 208 and securing the lymph node 208 in the pathology container 302. A further pathology container 302 may be inserted back into the suction dissector 110 and navigation to the next location of a lymph node 208 can be undertaken. In a typical procedure as many as twelve lymph nodes may need to be dissected from the patient to accurately assess the spread of the disease from the lesion.

As noted above, articulation of the gooseneck 202 may be achieved through the use of one or more pull wires 214 which extend the length of the shaft and are either mechanically or electromechanically linked to a control 132. In its simplest form control 132 acts on a single pull 214 wire to cause the gooseneck to articulate in a single direction. Alternatively, the control 132 may act on a two pull-wire system, where one pull wire is retracted and a second pull wire extends in a push-pull system to allow articulation in two opposing directions. Further, the control 132 may act on a four pull-wire system which allows for articulation in at least four orthogonal directions. The control 132 may energize one or more motors (not shown). The motors each act on a pair of pull wires 314 to achieve the desired articulation of the gooseneck 202.

As noted above control 133 may also be included on the handle 115. As with the pull wires 214 for articulation of the gooseneck 202, pull wires 216 may be employed to open and close the gripping fingers 206. On one embodiment movement of the pull wires 216 is a first direction forces the gripping fingers 206 apart so that the garter electrode 210 may be placed over the lymph node 208. As RF energy is applied to the garter electrode 210 and the tissue around the lymph node 208 is cut and cauterized, slow retraction of the gripping fingers 206 until the lymph node 208 is severed from the surrounding tissue. As with the pull wires 214, the pull wires 216 may be driven by one or more motors to extend or retract the gripping fingers 206.

Still further, the proximal end of the gripping fingers 206 may be affixed to the gooseneck 202, or directly to the shaft 120 in a manner that they are biased to a generally closed position. This may be achieved by bending of a proximal portion of the gripping fingers 206 such that the material they are formed of e.g., a metal such as aluminum or stainless steel imparts a spring force relative to the gooseneck 202 or the shaft 120 to force the distal ends of the gripping fingers 206 towards one another. This spring force may opposes to opening of the garter electrode 210 as the gripping fingers 206 are forced over the lymph node 208, as described above. The spring force of the material from which the gripping fingers 206 are formed may also be augmented by the spring force applied by the garter electrode 210. Still further, one or more springs 218 such as leaf springs, coil springs, or others may be placed at the connection of the gripping fingers 206 and the gooseneck 202 or shaft 120 to provide the biasing of the gripping fingers 206.

In accordance with a further embodiment of the disclosure the controls 131-133 are part of a robotic system and are not formed on the handle 115 but rather are part of a larger control panel, either hardware or software via, for example, a graphic user interface appearing on a screen in the surgical theater. Operation of the controls in the robotic system drive, articulate, and actuate the suction dissector 110 substantially in the same manner described above but without a user physically holding the handle 115 of the suction dissector 110.

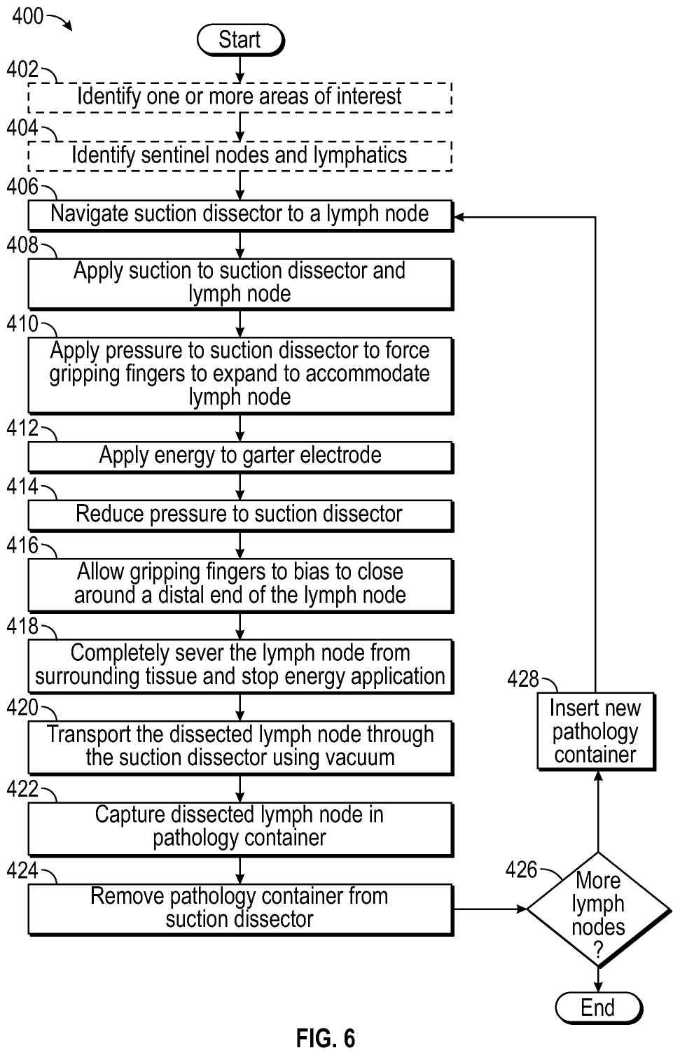

FIG. 6 depicts an exemplary method 400 a surgeon may follow when utilizing the suction dissector 110 in accordance with the disclosure. Option steps are noted with dashed borders, and the method may proceed without undertaking these steps in accordance with the disclosure. At step 402 one or more areas of interest may be identified for biopsy or analysis within the patient. These areas of interest may be identified via pre-procedural imaging such as computed tomography (CT) or magnetic resonance imaging (MRI) techniques or others. At step 404 the lymphatic system and sentinel lymph nodes are identified. As described elsewhere herein, once the target or lesion is identified, the lymphatic drainage from that lesion may be identified using a radioactive substance that may be detected to identify the sentinel nodes and other subsequent lymph nodes. Alternatively lymph nodes may be identified by imaging using ultrasound, CT or MRI imaging, or by observation by the surgeon trained in the physiology and anatomy of the patient. Having identified the location of the lymph nodes to be dissected, at step 406 the suction dissector 110 is navigated to the location of the lymph node 208. This navigation may be performed laparoscopically using and endoscope, endoluminally (e.g., navigation within the airways of the lungs or other lumens of the body) using for example a bronchoscope, or other methods suitable for placement of the distal end of the suction dissector 110 proximate the lymph node 208. As will be appreciated this navigation may be assisted by the articulation of the gooseneck 202 or robotic advancement of the suction dissector 110 as described above. Once proximate the lymph node 208 suction may be applied at step 408. Application of the suction assists in drawing the lymph node 208 into the gripping fingers 206 and shaft 120. At step 410 pressure may be applied to the proximal end of the suction dissector 110 such that the gripping fingers 206 are forced to widen around the lymph node 208. Again, this pressure may be applied by a robot and the opening of the gripping fingers 206 assisted by advancement of the pull wires 216 as described above. At step 412, with the pressure applied the garter electrode 210 is energized with a suitable RE energy (either monopolar or bipolar depending on the construction) and tissue around the lymph node 208 is cut and cauterized. This may be accomplished by activation of control 130 on handle 115. At step 414 upon reaching a desired depth, the pressure on the proximal end of the suction dissector 110 and therewith the pressure forcing the gripping fingers 206 to expand is relaxed. At step 416 the biasing force generated by the garter electrode 210 either alone or in combination with a spring force from the gripping fingers or an auxiliary spring affixed thereto cause the gripping fingers 206 to close around a distal end of the lymph node. This may further be accomplished in association with retraction of the pull wires 216 to close the gripping fingers. At step 418 the gripping fingers 206 completely sever the lymph node 208 from the surrounding tissue and the RE energy is ceased. At step 420, continued application of vacuum to the shaft 120 and gooseneck 202 cause the dissected lymph node to travel the length of the suction dissector 110. At step 422 the lymph node 208 is captured in the pathology container 302 and the vacuum is ceased. Once the dissected lymph node 208 is captured the pathology container 302 is removed from the suction dissector 110 at step 424. At step 426 a determination is made whether further lymph nodes 208 need to be dissected. If not then the procedure end, but if there are further lymph nodes 208 to dissect then a new pathology container 302 is inserted into the suction dissector 110 at step 428 and the method returns to step 406.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects.

We claim:

1. A suction dissector comprising:
a handle configured with a plurality of controls;
a vacuum source in fluid communication with the handle and operable with one of the plurality of controls;
a shaft in fluid communication with the vacuum source and connected to the handle;
a gooseneck formed of articulating joints and connected to a distal end of the shaft, the gooseneck configured to articulate in at least one direction;
a plurality of gripping fingers flexibly connected to a distal end of the gooseneck;
a garter electrode interconnecting the plurality of gripping fingers, the garter electrode being sized to be received over a lymph node and biasing the gripping fingers in a closed position;
an energy source electrically connected to the garter electrode, wherein the garter electrode is configured to cut and cauterize tissue in contact with the garter electrode;
a plurality of first pull wires configured to articulate the gooseneck and operable with one of the plurality of controls;
a plurality of second pull wires configured to move the gripping fingers from the closed position to an open position and operable with one of the plurality of controls; and
a pathology container in fluid communication with the vacuum source and the shaft, wherein upon dissection the lymph node by application of energy generated by the energy source to the garter electrode to cut and cauterize tissue surrounding the lymph node, the lymph node is transported along the gooseneck and shaft by the vacuum and received in the pathology container.

2. The suction dissector of claim 1, wherein the first and second pull wires are motor driven.

3. A suction dissector comprising:
a vacuum source;
a shaft in fluid communication with the vacuum source;
a gooseneck formed of a plurality of articulating joints and connected to a distal end of the shaft, the gooseneck configured to articulate in at least one direction;
a plurality of gripping fingers flexibly connected to a distal end of the gooseneck;
a garter electrode interconnecting the plurality of gripping fingers and sized to be received over a lymph node;
an energy source electrically connected to the garter electrode, wherein application of energy to the garter electrode cuts and cauterizes tissue in contact with the garter electrode; and
a pathology container in fluid communication with the vacuum source and the shaft, wherein upon dissection the lymph node is transported along the shaft by the vacuum and received in the pathology container.

4. The suction dissector of claim 3, further comprising at least one first pull wire to articulate the gooseneck.

5. The suction dissector of claim 4, wherein the at least one first pull wire is operably connected to motor to extend and retract the at least one first pull wire.

6. The suction dissector of claim 5, wherein the at least one pull wire is robotically actuated.

7. The suction dissector of claim 1, further comprising at least one second pull wire to open or close the of gripping fingers.

8. The suction dissector of claim 7, wherein the at least one second pull wire is operably connected to motor to extend and retract the at least one second pull wire.

9. The suction dissector of claim 8, wherein the at least one second pull wire is robotically actuated.

10. The suction dissector of claim 3, wherein the garter electrode applies a spring force against the gripping fingers to move the gripping fingers into a closed position.

11. The suction dissector of claim 3, wherein the gripping fingers are formed such that they apply a spring force relative to the shaft to move the gripping fingers into a closed position.

12. The suction dissector of claim 3, further comprising a spring associated with the gripping fingers to move the gripping fingers into a closed position.

13. The suction dissector of claim 3, wherein the pathology container is removable and replaceable.

14. A method of dissecting a lymph node comprising:
navigating a distal portion of a suction dissection catheter to a lymph node within a patient;
forcing one or more gripping fingers secured to a distal end of the suction dissection catheter over a portion of the lymph node;
applying a vacuum through a shaft of the suction dissection catheter;
applying energy from an energy source to a garter electrode connecting the one or more gripping fingers to tissue surrounding the lymph node such that the tissue surrounding the garter electrode is cut an cauterized;
dissecting the lymph node from the tissue surrounding the lymph node;
drawing the dissected lymph node through the shaft via the applied vacuum; and
capturing the dissected lymph node in a pathology container.

15. The method of claim 14, further comprising determining a location of one or more lymph nodes;

16. The method of claim 15, wherein the determining is via application of a radioactive substance to a lesion and observing transmission of the radioactive substance to one or more lymph nodes.

17. The method of claim 14, wherein the navigation is performed via a robotic interface with the suction dissection catheter.

18. The method of claim 14, wherein the energy is radio frequency energy.

19. The method of claim 14, wherein application of force on the gripping fingers causes them to expand to extend around the lymph node and easing of the force allows the garter electrode to retract the gripping fingers.

20. The method of claim 19, wherein a spring force associated with one or more of the garter electrode, a material of the gripping fingers, or a spring incorporated into the interface between the gripping fingers and a gooseneck at the end of the shaft urge the gripping fingers together.
